(19) United States
(12) Patent Application Publication
(10) Pub. No.: US 2024/0122535 A1
(43) Pub. Date: Apr. 18, 2024

(54) TECHNIQUES FOR PERSONALIZED WELLNESS ROUTINES USING WEARABLE DEVICE AND EXTERNAL DEVICE

(71) Applicant: Oura Health Oy, Oulu (FI)

(72) Inventors: Tuula-Liina Rees, Oulu (FI); Brian James Gilan, San Francisco, CA (US); Ralph Morales, Saratoga, CA (US); Geoffrey Hunter Wylde, San Francisco, CA (US); Yiien Liu, San Francisco, CA (US)

(21) Appl. No.: 18/488,276

(22) Filed: Oct. 17, 2023

Related U.S. Application Data
(60) Provisional application No. 63/416,794, filed on Oct. 17, 2022.

(51) Int. Cl. A61B 5/00; A61B 5/026; G16H 40/67

(52) U.S. Cl. CPC A61B 5/486; A61B 5/0261; A61B 5/6826; A61B 5/742; G16H 40/67

(57) ABSTRACT

Methods, systems, and devices for operating a user device are described. A user device may identify a health-related condition of a user based on a user input, PPG data for the user, or both. The user device may display instructions for performing a wellness routine that is configured to alleviate the health-related condition. The user device may acquire an additional user input, additional PPG data, or both, during a trial period for the wellness routine. The user device may display information associated with the wellness routine that is based on the additional user input, the additional PPG data, or both.

### CROSS REFERENCE

The present application for patent claims the benefit of U.S. Provisional Patent Application No. 63/416,794 by Rees et al., entitled “TECHNIQUES FOR PERSONALIZED WELLNESS ROUTINES USING WEARABLE DEVICE AND EXTERNAL DEVICE,” filed Oct. 17, 2022, which is assigned to the assignee hereof and expressly incorporated by reference herein.

### FIELD OF TECHNOLOGY

The following relates to wearable devices and data processing, including techniques for personalized wellness routines using a wearable device and an external device.

### BACKGROUND

Some wearable devices may be configured to collect data (e.g., physiological data) from users that allows associated user devices to provide insights into the health and well-being of the user. However, some insights provided by the user devices may not enable the user to improve their health and well-being. As such, techniques for improving the health and well-being of a user may be desired.

### BRIEF DESCRIPTION OF THE DRAWINGS

FIG. 1 illustrates an example of a system that supports techniques for personalized wellness routines using a wearable device and an external device in accordance with aspects of the present disclosure.

FIG. 2 illustrates an example of a system that supports techniques for personalized wellness routines using a wearable device and an external device in accordance with aspects of the present disclosure.

FIG. 3 illustrates an example of a system that supports techniques for personalized wellness routines using a wearable device and an external device in accordance with aspects of the present disclosure.

FIG. 4 illustrates examples of graphical user interfaces (GUIs) that support techniques for personalized wellness routines using a wearable device and an external device in accordance with aspects of the present disclosure.

FIG. 5 illustrates a block diagram of an apparatus that supports techniques for personalized wellness routines using a wearable device and an external device in accordance with aspects of the present disclosure.

FIG. 6 illustrates a block diagram of a wearable application that supports techniques for personalized wellness routines using a wearable device and an external device in accordance with aspects of the present disclosure.

FIG. 7 illustrates a diagram of a system including a device that supports techniques for personalized wellness routines using a wearable device and an external device in accordance with aspects of the present disclosure.

FIG. 8 illustrates a flowchart showing methods that support techniques for personalized wellness routines using a wearable device and an external device in accordance with aspects of the present disclosure.

### DETAILED DESCRIPTION

A user device may receive data, such as physiological data (e.g., photoplethysmogram (PPG) data), for a user from a wearable device coupled with the user device and may use the data to determine various health metrics for the user. For example, the user device may use the physiological (e.g., PPG) data for the user to determine the quality of sleep of the user. The user device may provide the information to the user so that the user is aware of the health metrics. However, some user devices may not provide any information about how the user can improve the health metrics or health-related conditions associated with the health metrics. In other words, some wearable devices and user devices may be used to report physiological data to the user, but may not provide any guidance as to how the user can improve their physiological data and overall health.

According to the techniques described herein, a user device may help a user improve health-related conditions by suggesting a personalized (e.g., user-specific) wellness routine that involves the use of one or more external devices and that is targeted to the health-related conditions of the user. The user device may identify the health-related condition of the user, and/or generate the wellness routine, based on user inputs received from the user, physiological data received from a wearable device of the user, or both.

The user device may monitor the health metrics of the user during a trial period for the wellness routine and may determine the efficacy of the wellness routine. For example, the user device may determine the efficacy of the wellness routine by comparing the trial-period health metrics with baseline health metrics collected before the trial period. After the trial period for the wellness routine, the user device may provide an indication of the efficacy of the wellness routine to the user so that the user can evaluate the merits (e.g., efficacy) of the wellness routine. The user device may also suggest modifications to the wellness routine to improve the efficacy of the wellness routine.

Aspects of the disclosure are initially described in the context of systems supporting physiological data collection from users via wearable devices. Additional aspects of the disclosure are described in the context of graphical user interfaces (GUIs). Aspects of the disclosure are further illustrated by and described with reference to apparatus diagrams, system diagrams, and flowcharts that relate to techniques for personalized wellness routines using a wearable device and an external device.

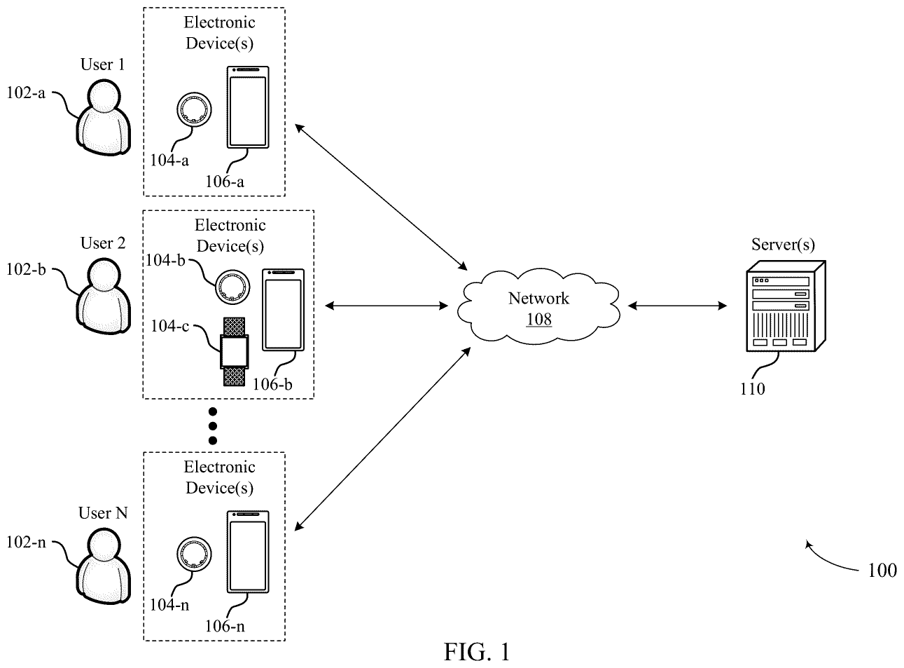

FIG. 1 illustrates an example of a system 100 that supports techniques for personalized wellness routines using a wearable device and an external device in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as light-emitting diodes (LEDs) (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks”, 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for improving one or more health-related conditions of a user 102. For example, a user device 106 may generate a personalized wellness routine for a user 102 that involves the use of one or more external devices and that is designed to alleviate one or more health-related conditions of the user 102. The wellness routine may also involve the use of an exercise routine, a diet, a meditation routine, a supplement routine (e.g., vitamin supplements), a stretching routine, a medication routine, instructions for performing cognitive behavioral therapy, or any combination thereof. The user device 106 may identify the health related-condition(s) based on a user input associated with the health-condition(s) (e.g., an indication of one or more pain points), based on physiological data (e.g., PPG data) received from a wearable device 104 of the user, or both. The user device 106 may also use the user input, PPG data, or both, to design the wellness routine.

In some examples, the user device 106 may determine an efficacy of the wellness routine (e.g., in terms of ameliorating the health-related condition(s) of the user 102) based on additional user input associated with the health-condition(s), based on additional PPG data from a wearable device 104 of the user, or both. The additional user input, the additional PPG data, or both, may be acquired during the trial period of the wellness routine, that may include a time interval that the wellness routine is performed, a time interval after performance of the wellness routine, or both. The user device 106 may display the efficacy of the wellness routine (e.g., how effective the wellness routine is/was in alleviating health-related conditions and/or changing the user's physiological data) so that the user can determine whether to continue to practice the wellness routine. Additionally or alternatively, the user device 106 may use the efficacy of the wellness routine as a basis to improve the wellness routine (e.g., by modifying one or more parameters of the wellness routine).

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

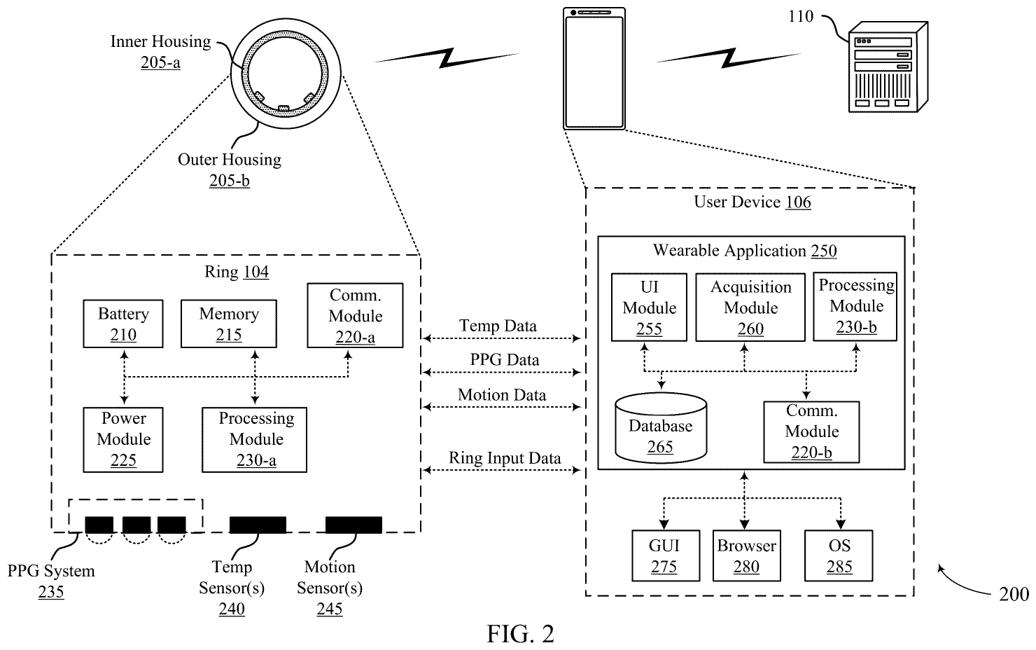

FIG. 2 illustrates an example of a system 200 that supports techniques for personalized wellness routines using a wearable device and an external device in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels, and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, PPG data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using a clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, that may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include LEDs. The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and Readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

According to the techniques described herein, the user device 106 may identify one or more health-related conditions of a user and determine a personalized wellness routine for alleviating the one or more health-related conditions. For example, the user device 106 may identify that the user is experiencing muscle pain, body tension, circulation issues, sleep issues, headaches, or body aches, mental health-related issues, cardiovascular issues, among other health-related conditions, and may recommend one or more personalized wellness routines configured to alleviate the health-related conditions.

The user device 106 may identify the one or more health-related conditions of the user based on a user input from the user, based on physiological data (e.g., heart rate values, IBI values, HRV values, respiratory rate values, oxygen saturation, temperature, motion data, sleep data, PPG data) received from the wearable device 104, or both. For example, the user device 106 may determine that a user has back pain based on a user input indicating as much. As another example, the user device 106 may determine that a user is experiencing poor sleep quality based on sleep data from the wearable device 104. As another example, the user device 106 may determine that a user is experiencing high levels of stress based on a user input from the user (e.g., indicating anxiety) and based on heart-related health metrics (e.g., heart rate, HRV) received from the wearable device 104.

After identifying a health-related condition, the user device 106 may determine a wellness routine that is configured to alleviate the health-related condition and that uses one or more external devices. For example, the user device 106 may determine a wellness routine for improving back pain that involves the use of a massage device, a percussive therapy device, a wearable vibrating thermal device, a foam roller (e.g., a vibrating foam roller), a cryotherapy device, or any combination thereof. As another example, the user device 106 may determine a wellness routine for improving sleep quality that involves blue light-blocking glasses, a weighted blanked, a sleep mask (e.g., a vibrating sleep mask), an electroshock therapy device (e.g., a neuromuscular electrical stimulation (NMES) device, a transcutaneous electrical nerve stimulation (TENS) device), a meditation routine, a cognitive behavioral therapy routine, or any combination thereof. As another example, the user device 106 may determine a wellness routine for reducing stress that involves a massage device, a vibrating sleep mask, a heating device, an infrared sauna, a hydrating facial, an exercise routine, or any combination thereof.

In some examples, the user device 106 may generate multiple candidate wellness routines from which the user 102 may select a wellness routine for trial. For example, the user device 106 may display a prompt that includes the candidate wellness routines and, in response to displaying the prompt, may receive a selection of a wellness routine from the candidate wellness routines (e.g., the user may elect to participate in one of the recommended wellness routines). The user device 106 may display a set of instructions for the selected wellness routine to the user so that the user can implement the selected wellness routine.

In some examples, a wellness routine generated by a user device 106 may be based on information associated with other users with health-related conditions similar to the health-related condition of the user. For example, the user device 106 may generate a wellness routine based on efficacy metrics for other wellness routines attempted by other users to alleviate health-related conditions similar to the health-related condition of the user. The user device 106 may estimate or predict an efficacy metric for the suggested wellness routine(s) based on the efficacy metrics for the other wellness routines. In some examples, the efficacy metric estimated for a suggested wellness routine may be weighted based on the similarities between personal characteristics (e.g., gender, age, weight, similar physiological data) of the other users and the user 102. The user device 106 may display the efficacy metrics for candidate wellness routines so that the user 102 can use the efficacy metrics as a basis for selecting a wellness routine for trial.

In some examples, the user device 106 may determine the actual efficacy of the selected wellness routine, in terms of ameliorating the health-related conditions of the user 102, so that the wellness routine can be improved (e.g., by modifying one or more parameters of the wellness routine) and/or so that the user 102 can evaluate the merits of the wellness routine. The actual efficacy of a wellness routine may differ from the predicated efficacy for the wellness routine. The user device 106 may determine the actual efficacy of the selected wellness routine based physiological (e.g., PPG) data acquired during a trial period for the wellness routine, based on a user input (also acquired during the trial period) associated with the health-related condition (e.g., an indication of the severity of the health-related condition relative to a baseline severity), or both. For example, throughout the trial period for a wellness routine intended to alleviate back pain, the user may input “tags” indicating their subjective severity of back pain, where the inputted tags may be used to evaluate the efficacy of the wellness routine in alleviating back pain. In other examples, throughout the trial period for a wellness routine, the user may input “tags” indicating attendance at a cryotherapy session, where the inputted tags may be used to tailor recommendations for future wellness routines based on user preference, wellness routine patterns, and efficacy of the wellness routine.

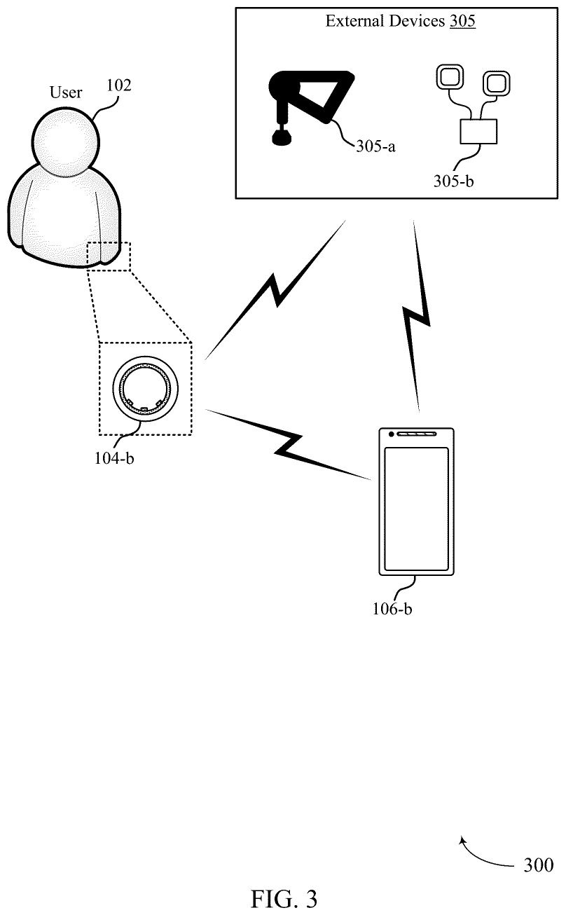

FIG. 3 illustrates an example of a system 300 that supports techniques for personalized wellness routines using a wearable device and an external device in accordance with aspects of the present disclosure. The system 300 may include a wearable device 104-a and a user device 106-a, that may be examples of corresponding devices as described in FIGS. 1 and 2. The system 300 may also include one or more external devices 305. The wearable device 104-a, the user device 106-a, and the external device 305 may be configured to communicate (e.g., wirelessly) various information that facilitates the configuration of wellness routines that are specific to the user 102. Examples of external devices 305 include a massage device, a percussive therapy device, a thermally-controlled device, a weighted blanket, blue light-blocking glasses, an electroshock therapy device, and a sleep mask. In some cases, examples of external devices 305 may include a cryotherapy device, a red light therapy device, an infrared sauna, a compression device, an intravenous (IV) device (e.g., to perform IV drip therapy, Nicotinamide adenine dinucleotide (NAD) IV therapy, and the like), an intramuscular shot, a hyperbaric oxygen therapy device (e.g., to perform mild hyperbaric oxygen therapy), a facial procedure (e.g., oxygen facial, vitamin C facial, hydrating hyaluronic facial, and the like), biomarker assessment device, or a combination thereof. In such cases, the external devices 305 may be configured to perform one or more user-perceptible actions associated with one or more wellness routines that are performable by the user. For example, one or more user-perceptible actions may include, but are not limited to, a massage or percussive intensity, a massage or percussive pattern, a temperature, a periodicity, a voltage or current, and the like.

The user device 106-b may identify a health-related condition of the user 102 based on physiological data received from the user device 104-a, based on user input information from the user 102, or both. The physiological data may be an example of PPG data. For example, the user device 106-b may determine that the user 102 is experiencing back pain based on the user 102 providing an indication of back pain (e.g., via a GUI), based on the wearable device 104-d indicating health metrics associated with chronic back pain (e.g., poor quality sleep, limited activity, low HRV), or both. Examples of health-related conditions include muscle pain, body tension or tightness, circulation issues, issues associated with sleep (e.g., insomnia, sleep apnea, and the like), headaches, and back aches. In some cases, examples of health-related conditions may include hypertension, one or more mental health-related conditions, one or more cardiovascular conditions, or a combination thereof. In some examples, the user device 106-b may use machine learning to identify health-related conditions of the user 102. In such examples, the user device 106-b may autonomously (e.g., based on physiological data and independent of user input from the user 102) identify a health-related condition of the user 102 and suggest a personalized wellness routine for alleviating the health-related condition. In some cases, the use of machine learning techniques may enable automatic “pain point” identification/suggestions.

For example, the system 300 may input the PPG data into the machine learning classifier. In such cases, in response to inputting the baseline PPG data into the machine learning classifier, the system 300 may identify one or more health-related conditions associated with the user. In some cases, the system 300 may input the user input into the machine learning classifier and one or more health-related conditions associated with the user in response to (e.g., after) inputting the PPG data, the user input, or both, into the machine learning classifier.

After identifying the health-related condition of the user 102, the user device 106-b may determine one or more candidate wellness routines configured to alleviate the health-related condition. For example, the user device 106-b may determine a first candidate wellness routine (Routine A) that includes use of the percussive therapy device 305-a and use of the electroshock therapy device 305-b. The user device 106-b may also determine a second candidate wellness routine (Routine B) that includes use of a foam roller and use of the percussive therapy device 305-a. In some examples, the candidate wellness routines may be determined based on predicted efficacy metrics for the candidate wellness routines that are in turn are based on actual efficacy metrics for the candidate wellness routines (or similar wellness routines) when used by other users with similar health-related conditions as the user 102. Additionally or alternatively, the efficacy metric for a candidate wellness routine may be based on the history of previous wellness routines performed by the user (e.g., routine history). For instance, the user device 106-b may determine or adjust the efficacy metric predicted for a candidate wellness routine based on the efficacy metrics of previous wellness routines performed by the user. In some cases, the system may identify the one or more candidate wellness routines based on inputting the PPG data, the user input, or both into the machine learning classifier.

A wellness routine may involve the use of an external device 305 and may include instructions on how to perform the wellness routine. For example, the instructions for a wellness routine may indicate how to use or apply the external device 305, the regimen for using the external device 305 (e.g., the quantity of times the external device 305 should be used per day or per week), the operational settings for the external device 305 (e.g., the pattern type, the heat level, the vibration level, the voltage level, the current level, the intensity level), or any combination thereof.

In addition to external devices 305, the wellness routine may involve the use of an exercise routine, a diet, a meditation routine, a supplement routine (e.g., vitamin routine), a stretching routine, a medication routine, a cognitive behavioral therapy routine, or any combination thereof.

The user device 106-b may use a GUI to display an indication of the candidate wellness routines for selection by the user 102, that may include displaying respective monikers for the wellness routines, displaying an indication of the external devices 305 involved in the wellness routines, displaying respective instructions for the wellness routines, or any combination thereof, among other examples. In some examples, the user device 106-b may display the predicted efficacy metrics for the candidate wellness routines. The user device 106-b may determine and display the candidate wellness routines in response to a request received from the user 102 or autonomously (e.g., based on identifying the health-related condition, independent of a request from the user 102).

After displaying the candidate wellness routines, the user device 106-b may receive an indication of the wellness routine selected by the user 102. If not already determined, the user device 106-b may determine the instructions for the wellness routine. For instance, the user device 106-b may determine a frequency for performing the wellness routine, a duration for performing the wellness routine, instructions for an exercise routine, instructions for a diet, instructions for performing a meditation, instructions for a supplement routine, instructions for a stretching routine, instructions for a medication routine, instructions for performing cognitive behavioral therapy, or any combination thereof. Additionally or alternatively, the user device 106-b may determine instructions for operating the external devices 305.

For example, the user device 106-b may determine one or more operational settings for the external devices 305 based on the personal characteristics of the user 102 (e.g., height, weight, gender), the user input from the user 102, the physiological data from the wearable device 104-b, or both. In some examples, the user device 106-b may transmit a signal to the external device(s) 305 (and/or to a user device/application associated with the external device(s) 305) indicating the operational settings for the respective external device(s) 305.

The user device 106-b may collect data during a trial period for the wellness routine so that the user device 106-b can determine an actual efficacy metric for the wellness routine. For example, the user device 106-b may receive additional physiological data collected via the wearable device 104-b during the trial period for comparison with baseline physiological data collected before the trial period. Additionally or alternatively, the user device 106-b and/or wearable device 104-b may receive additional user input data (e.g., tags, such as tags associated with a severity of symptoms of the health-related conditions) during the trial period for comparison with baseline user input data collected before the trial period. The trial period may include a first time interval that the wellness routine is performed by the user 102, a second time interval subsequent to performance of the wellness routine (e.g., subsequent to the first time interval), or both.

In some examples, the user device 106-b may receive information related to the wellness routine from the external device(s) 305 involved in the wellness routine. For example, an external device 305 may report to the user device 106-b information about the use of the external device 305, such as the frequency of use, the length of use, the operational settings during use, or any combination thereof, among other examples. The user device 106-b may use the information from the external device 305 to determine the user's compliance (e.g., adherence) with the wellness routine. Additionally or alternatively, the user device 106-b may receive physiological data measured by the external device(s) 305. In such examples, the user device 106-b may use the physiological data received from the external device(s) 305 as a basis for calculating an actual efficacy metric for the wellness routine, as a basis for modifying the wellness routine, or both. Physiological data received from the external devices 305 may be used in addition to, or in the alternate to, physiological data acquired by the wearable device 104-a. For example, referring to the system 300, the wearable device 104-a may acquire PPG data from the user using the one or more light-emitting components and one or more light-receiving components.

Before, after, or during the trial period, the user device 106-b may provide information associated with the wellness routine to the user 102. For example, the user device 106-b may use a GUI to display the efficacy metric determined for the wellness routine. Additionally or alternatively, the user device 106-b may display one or more changes between the baseline physiological data and the trial period physiological data. For example, the user device 106-b may display a relative relationship between a health metric determined for the user 102 before and after performance of the wellness routine (e.g., an indication that the user's Sleep Score has improved by x points). Additionally or alternatively, the user device 106-b may display information associated with an additional user input received in response to the wellness routine. For example, the user device 106-b may display an indication that the user 102 tagged “back pain” less frequently on the days or weeks the user 102 performed the wellness routine. The user device 106-b may provide information associated with the wellness routine to the user 102 before the trial period, during the trial period, after the trial period, or any combination thereof.

In some examples, the user device 106-b may display one or more suggested modifications for the wellness routine, that may be determined based on the data acquired during the trial period. In some examples, the user device 106-b may display a message indicating the compliance/adherence of the user 102 in performing the wellness routine (e.g., based on information from the external device(s) 305).

Thus, the user device 106-b may generate a wellness routine to alleviate a health-related condition identified for the user 102.

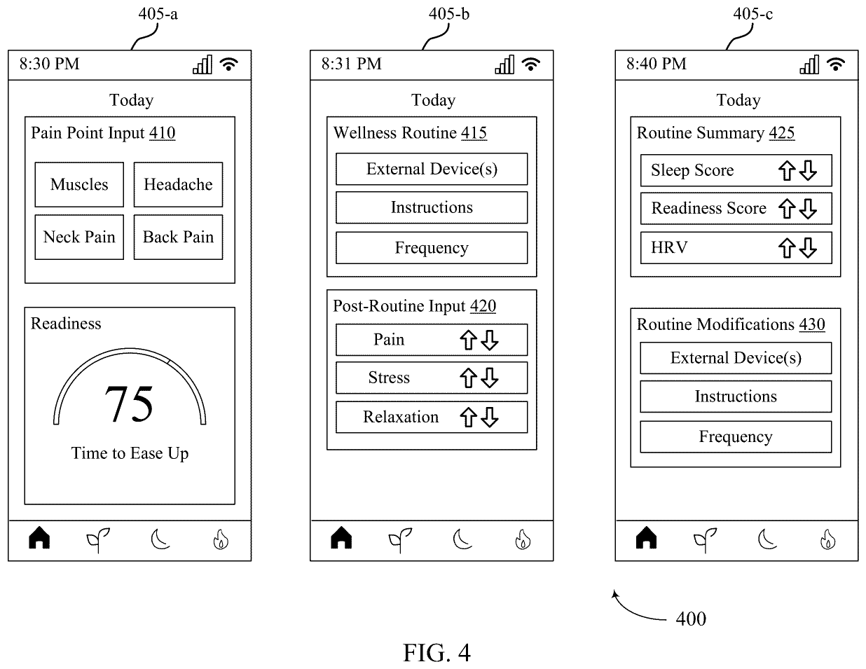

FIG. 4 illustrates examples of GUIs 400 that support techniques for personalized wellness routines using a wearable device and an external device in accordance with aspects of the present disclosure. The GUIs 405 may be examples of GUIs displayed by a user device 106 as described herein. The GUIs 405 may facilitate the determination, use, and monitoring of a personalized wellness routine that incorporates the use of one or more external devices.

The GUI 405-a may display an input prompt 410 that provides different options for a pain point (e.g., health-related conditions) that are selectable by the user. Thus, the selected pain point may be an example of a user input as described herein. The user device may identify a health-related condition of the user based on the pain point selected by the user, based on physiological data for the user, or both. In some examples, the GUI 405-a may also display a severity prompt that provides different severity levels for the pain point (e.g., so that the user device can evaluate the efficacy of a wellness routine configured to alleviate the pain point). In some examples, the user device may determine the severity of the health-related condition based on the physiological data for the user.

The GUI 405-b may display wellness routine information 415 for a wellness routine that is configured to alleviate the health-related condition identified for the user. In some examples, the GUI 405-b may display the wellness routine information 415 based on a selection of the corresponding wellness routine from a set of candidate wellness routines. The wellness routine information 415 may include the external device(s) involved in the wellness routine, the instructions for the wellness routine (e.g., instructions for using the external device(s), a duration for performing the wellness routine, instructions for an exercise routine, instructions for a diet, instructions for performing a meditation, instructions for a supplement routine, instructions for a stretching routine), a frequency for performing the wellness routine, instructions for a medication routine, instructions for performing cognitive behavioral therapy, or any combination thereof.

The GUI 405-b may also display a post-routine input prompt 420 that provides different options for subjective assessments that are selectable by the user. For example, the post-routine input prompt 420 may include an option for indicating the relative pain felt by the user before performing the wellness routine and after performing the wellness routine. As another example, the post-routine input prompt 420 may include an option for indicating the relative stress felt by the user before performing the wellness routine and after performing the wellness routine. As another example, the post-routine input prompt 420 may include an option for indicating the relative relaxation felt by the user before performing the wellness routine and after performing the wellness routine. The user device 106-b may use the data from the post-routine input prompt 420 to determine the efficacy of the wellness routine.

The GUI 405-c may display a routine summary 425 for the wellness routine. The routine summary 425 may indicate whether various health metrics for the user have improved since performance of the wellness routine. For instance, the routine summary 425 may indicate whether the Sleep Score of the user has improved (e.g., based on a comparison of an initial Sleep Score acquired for the user before performance of the wellness routine and an updated Sleep Score acquired for the user after performance of the wellness routine). Additionally or alternatively, the routine summary 425 may indicate whether the Readiness Score of the user has improved (e.g., based on a comparison of an initial Readiness Score acquired for the user before performance of the wellness routine and an updated Readiness Score acquired for the user after performance of the wellness routine). Additionally or alternatively, the routine summary 425 may indicate whether the HRV of the user has improved (e.g., based on a comparison of an initial HRV acquired for the user before performance of the wellness routine and an updated HRV acquired for the user after performance of the wellness routine). The user device 106-b may use the data from the routine summary 425 to determine the efficacy of the wellness routine.

The GUI 405-c may also display one or more routine modifications 430 for the wellness routine. The routine modifications may include a change in the external device(s) involved in the wellness routine, a change in the instructions for the wellness routine, a change in the frequency that the external device(s) are to be used, or any combination thereof, among other options. The routine modifications may be determined based on an efficacy metric for the wellness routine, that in turn may be based on the subjective assessments received via the post-routine input prompt 420, the relative health metrics indicated by the routine summary 425, or both.

Thus, the GUIs 405 may facilitate the determination, use, and monitoring of a personalized wellness routine that incorporates the use of one or more external devices.

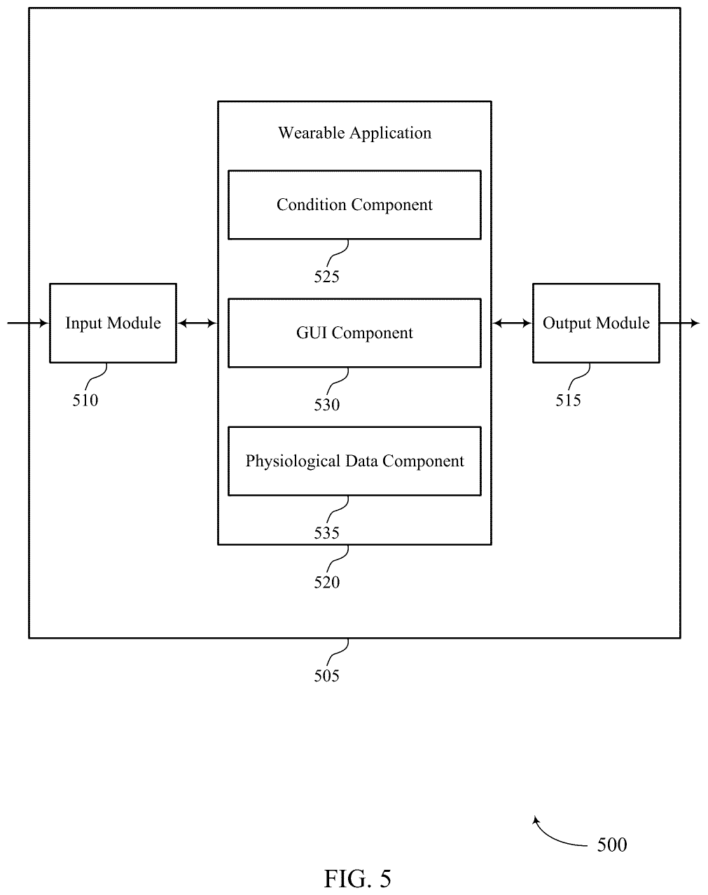

FIG. 5 illustrates a block diagram 500 of a device 505 that supports techniques for personalized wellness routines using a wearable device and an external device in accordance with aspects of the present disclosure. The device 505 may include an input module 510, an output module 515, and a wearable application 520. The device 505 may also include a processor. Each of these components may be in communication with one another (e.g., via one or more buses).

The input module 510 may provide a means for receiving information such as packets, user data, control information, or any combination thereof associated with various information channels (e.g., control channels, data channels, information channels related to illness detection techniques). Information may be passed on to other components of the device 505. The input module 510 may utilize a single antenna or a set of multiple antennas.

The output module 515 may provide a means for transmitting signals generated by other components of the device 505. For example, the output module 515 may transmit information such as packets, user data, control information, or any combination thereof associated with various information channels (e.g., control channels, data channels, information channels related to illness detection techniques). In some examples, the output module 515 may be co-located with the input module 510 in a transceiver module. The output module 515 may utilize a single antenna or a set of multiple antennas.

For example, the wearable application 520 may include a condition component 525, a GUI component 530, a physiological data component 535, or any combination thereof. In some examples, the wearable application 520, or various components thereof, may be configured to perform various operations (e.g., receiving, monitoring, transmitting) using or otherwise in cooperation with the input module 510, the output module 515, or both. For example, the wearable application 520 may receive information from the input module 510, send information to the output module 515, or be integrated in combination with the input module 510, the output module 515, or both to receive information, transmit information, or perform various other operations as described herein.

The condition component 525 may be configured as or otherwise support a means for identifying one or more health-related conditions associated with a user based at least in part on PPG data collected via a wearable device, a user input received via a user device, or both. The GUI component 530 may be configured as or otherwise support a means for transmitting an instruction to cause a GUI of the user device to display a set of instructions for performing a wellness routine configured to alleviate the one or more health-related conditions, wherein the wellness routine is determined based at least in part on the one or more health-related conditions and the PPG data associated with the user, and wherein the set of instructions comprise one or more instructions for using one or more external devices in accordance with the wellness routine. The physiological data component 535 may be configured as or otherwise support a means for receiving additional PPG data acquired by the wearable device during a first time interval that the wellness routine is performed, during a second time interval subsequent to performance of the wellness routine, or both. The GUI component 530 may be configured as or otherwise support a means for transmitting an additional instruction to cause the GUI of the user device to display information associated with the wellness routine, wherein the information comprises one or more changes between the PPG data and the additional PPG data, information associated with an additional user input received in response to the wellness routine, or both.

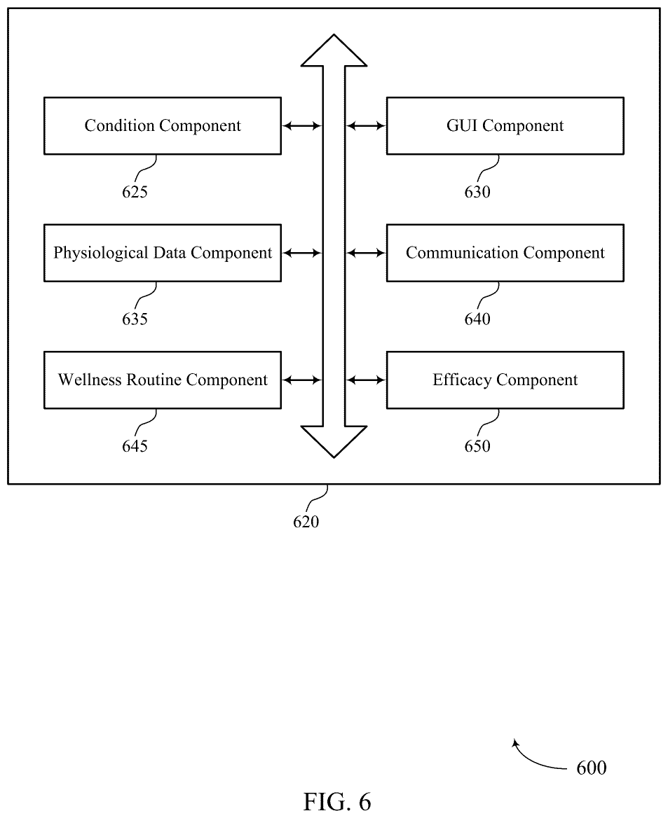

FIG. 6 illustrates a block diagram 600 of a wearable application 620 that supports techniques for personalized wellness routines using a wearable device and an external device in accordance with aspects of the present disclosure. The wearable application 620 may be an example of aspects of a wearable application or a wearable application 520, or both, as described herein. The wearable application 620, or various components thereof, may be an example of means for performing various aspects of techniques for personalized wellness routines using a wearable device and an external device as described herein. For example, the wearable application 620 may include a condition component 625, a GUI component 630, a physiological data component 635, a communication component 640, a wellness routine component 645, an efficacy component 650, or any combination thereof. Each of these components may communicate, directly or indirectly, with one another (e.g., via one or more buses).

The condition component 625 may be configured as or otherwise support a means for identifying one or more health-related conditions associated with a user based at least in part on PPG data collected via a wearable device, a user input received via a user device, or both. The GUI component 630 may be configured as or otherwise support a means for transmitting an instruction to cause a GUI of the user device to display a set of instructions for performing a wellness routine configured to alleviate the one or more health-related conditions, wherein the wellness routine is determined based at least in part on the one or more health-related conditions and the PPG data associated with the user, and wherein the set of instructions comprise one or more instructions for using one or more external devices in accordance with the wellness routine. The physiological data component 635 may be configured as or otherwise support a means for receiving additional PPG data acquired by the wearable device during a first time interval that the wellness routine is performed, during a second time interval subsequent to performance of the wellness routine, or both. In some examples, the GUI component 630 may be configured as or otherwise support a means for transmitting an additional instruction to cause the GUI of the user device to display information associated with the wellness routine, wherein the information comprises one or more changes between the PPG data and the additional PPG data, information associated with an additional user input received in response to the wellness routine, or both.

In some examples, the GUI component 630 may be configured as or otherwise support a means for transmitting an instruction to cause the GUI of the user device to display a prompt including one or more candidate wellness routines configured to alleviate the one or more health-related conditions, the one or more candidate wellness routines including the wellness routine. In some examples, the communication component 640 may be configured as or otherwise support a means for receiving, via the user device based at least in part on displaying the prompt, a selection of the wellness routine from the one or more candidate wellness routines, wherein transmitting an instruction to cause the GUI of the user device to display the set of instructions for the wellness routine is based at least in part on receiving the selection.

In some examples, the efficacy component 650 may be configured as or otherwise support a means for determining one or more estimated efficacy metrics associated with the one or more candidate wellness routines, wherein the one or more candidate wellness routines are displayed based at least in part on the one or more estimated efficacy metrics.

In some examples, the wellness routine component 645 may be configured as or otherwise support a means for identifying the one or more candidate wellness routines, identifying the one or more estimated efficacy metrics, or both, based at least in part on a plurality of additional health-related conditions associated with a plurality of additional users, and a plurality of additional wellness routines performed by the plurality of additional users.

In some examples, the one or more instructions for using the one or more external devices comprise an indication of one or more operational settings associated with the one or more external devices.

In some examples, the wellness routine component 645 may be configured as or otherwise support a means for determining one or more operational settings associated with the one or more external devices based at least in part on the wellness routine. In some examples, the communication component 640 may be configured as or otherwise support a means for transmitting a signal to the one or more external devices, an additional user device associated with the one or more external devices, or both, wherein the signal comprises an additional instruction for the one or more external devices to operate in accordance with the one or more operational settings.

In some examples, the one or more operational settings comprise a massage or percussive intensity, a massage or percussive pattern, a temperature, a periodicity, a voltage or current, or any combination thereof.

In some examples, the wellness routine component 645 may be configured as or otherwise support a means for selectively modifying one or more parameters associated with the wellness routine based at least in part on the additional PPG data, the additional user input received in response to the wellness routine, or both. In some examples, the GUI component 630 may be configured as or otherwise support a means for transmitting an instruction to cause the GUI of the user device to display an additional set of instructions for performing the modified wellness routine.

In some examples, the set of instructions comprise a frequency for performing the wellness routine, a duration for performing the wellness routine, instructions for an exercise routine, instructions for a diet, instructions for performing a meditation, instructions for a supplement routine, instructions for a stretching routine, instructions for a medication routine, instructions for performing cognitive behavioral therapy, or any combination thereof.

In some examples, the one or more external devices comprise a massage device, a percussive therapy device, a thermally-controlled device, a weighted blanket, blue light-blocking glasses, an electroshock therapy device, a sleep mask, a cryotherapy device, a red light therapy device, an infrared sauna, a compression device, an intravenous device, an intramuscular shot, a hyperbaric oxygen therapy device, a facial procedure, or any combination thereof.

In some examples, the one or more health-related conditions comprise muscle pain, body tension or tightness, circulation issues, issues associated with sleep, headaches, back aches, hypertension, one or more mental health-related conditions, one or more cardiovascular conditions, or any combination thereof.

In some examples, the wearable device comprises a wearable ring device.

In some examples, the PPG data, the additional PPG data, or both, is acquired by the wearable device based on arterial blood flow, capillary blood flow, arteriole blood flow, or a combination thereof.

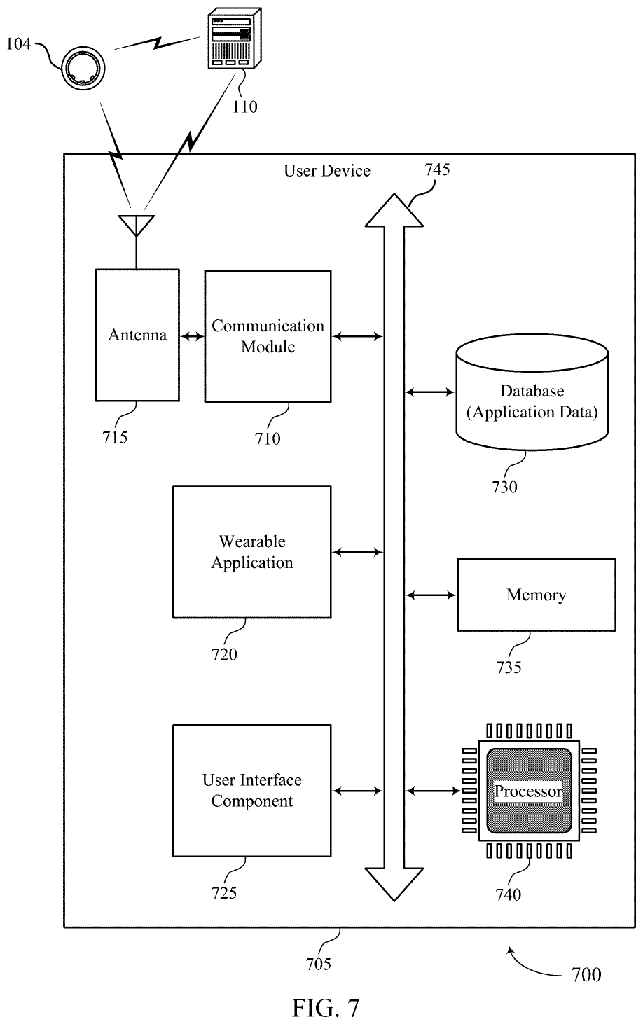

FIG. 7 illustrates a diagram of a system 700 including a device 705 that supports techniques for personalized wellness routines using a wearable device and an external device in accordance with aspects of the present disclosure. The device 705 may be an example of or include the components of a device 505 as described herein. The device 705 may include an example of a user device 106, as described previously herein. The device 705 may include components for bi-directional communications including components for transmitting and receiving communications with a wearable device 104 and a server 110, such as a wearable application 720, a communication module 710, an antenna 715, a user interface component 725, a database (application data) 730, a memory 735, and a processor 740. These components may be in electronic communication or otherwise coupled (e.g., operatively, communicatively, functionally, electronically, electrically) via one or more buses (e.g., a bus 745).

The communication module 710 may manage input and output signals for the device 705 via the antenna 715. The communication module 710 may include an example of the communication module 220-b of the user device 106 shown and described in FIG. 2. In this regard, the communication module 710 may manage communications with the ring 104 and the server 110, as illustrated in FIG. 2. The communication module 710 may also manage peripherals not integrated into the device 705. In some cases, the communication module 710 may represent a physical connection or port to an external peripheral. In some cases, the communication module 710 may utilize an operating system such as iOS®, ANDROID®, MS-DOS®, MS-WINDOWS®, OS/2®, UNIX®, LINUX®, or another known operating system. In other cases, the communication module 710 may represent or interact with a wearable device (e.g., ring 104), modem, a keyboard, a mouse, a touchscreen, or a similar device. In some cases, the communication module 710 may be implemented as part of the processor 740. In some examples, a user may interact with the device 705 via the communication module 710, user interface component 725, or via hardware components controlled by the communication module 710.

In some cases, the device 705 may include a single antenna 715. However, in some other cases, the device 705 may have more than one antenna 715, that may be capable of concurrently transmitting or receiving multiple wireless transmissions. The communication module 710 may communicate bi-directionally, via the one or more antennas 715, wired, or wireless links as described herein. For example, the communication module 710 may represent a wireless transceiver and may communicate bi-directionally with another wireless transceiver. The communication module 710 may also include a modem to modulate the packets, to provide the modulated packets to one or more antennas 715 for transmission, and to demodulate packets received from the one or more antennas 715.

The user interface component 725 may manage data storage and processing in a database 730. In some cases, a user may interact with the user interface component 725. In other cases, the user interface component 725 may operate automatically without user interaction. The database 730 may be an example of a single database, a distributed database, multiple distributed databases, a data store, a data lake, or an emergency backup database.

The memory 735 may include RAM and ROM. The memory 735 may store computer-readable, computer-executable software including instructions that, when executed, cause the processor 740 to perform various functions described herein. In some cases, the memory 735 may contain, among other things, a BIOS that may control basic hardware or software operation such as the interaction with peripheral components or devices.

The processor 740 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 740 may be configured to operate a memory array using a memory controller. In other cases, a memory controller may be integrated into the processor 740. The processor 740 may be configured to execute computer-readable instructions stored in a memory 735 to perform various functions (e.g., functions or tasks supporting a method and system for sleep staging algorithms).

For example, the wearable application 720 may be configured as or otherwise support a means for identifying one or more health-related conditions associated with a user based at least in part on PPG data collected via a wearable device, a user input received via a user device, or both. The wearable application 720 may be configured as or otherwise support a means for transmitting an instruction to cause a GUI of the user device to display a set of instructions for performing a wellness routine configured to alleviate the one or more health-related conditions, wherein the wellness routine is determined based at least in part on the one or more health-related conditions and the physiological data associated with the user, and wherein the set of instructions comprise one or more instructions for using one or more external devices in accordance with the wellness routine. The wearable application 720 may be configured as or otherwise support a means for receiving additional PPG data acquired by the wearable device during a first time interval that the wellness routine is performed, during a second time interval subsequent to performance of the wellness routine, or both. The wearable application 720 may be configured as or otherwise support a means for transmitting an additional instruction to cause the GUI of the user device to display information associated with the wellness routine, wherein the information comprises one or more changes between the PPG data and the additional PPG data, information associated with an additional user input received in response to the wellness routine, or both.

By including or configuring the wearable application 720 in accordance with examples as described herein, the device 705 may support techniques for improved health and wellness for a user.

The wearable application 720 may include an application (e.g., “app”), program, software, or other component that is configured to facilitate communications with a ring 104, server 110, other user devices 106, and the like. For example, the wearable application 720 may include an application executable on a user device 106 that is configured to receive data (e.g., physiological data) from a ring 104, perform processing operations on the received data, transmit and receive data with the servers 110, and cause presentation of data to a user 102.

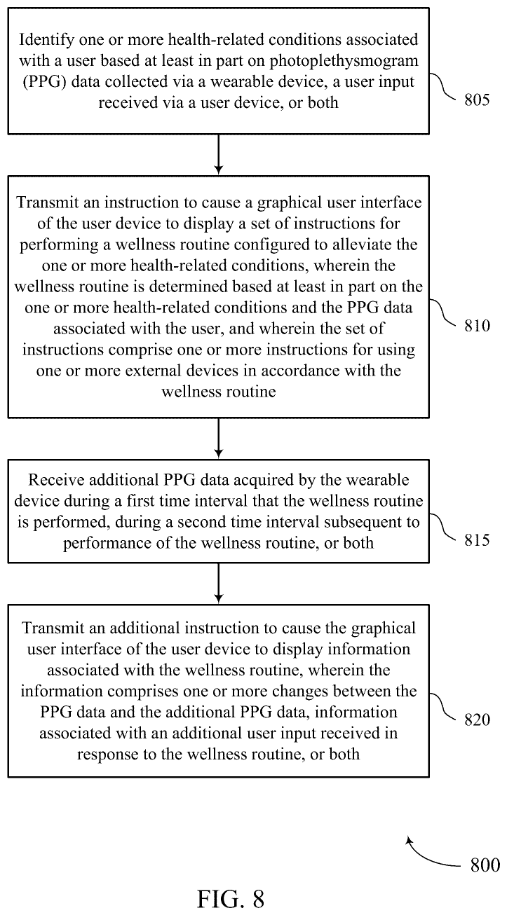

FIG. 8 illustrates a flowchart showing a method 800 that supports techniques for personalized wellness routines using a wearable device and an external device in accordance with aspects of the present disclosure. The operations of the method 800 may be implemented by a user device or its components as described herein. For example, the operations of the method 800 may be performed by a user device as described with reference to FIGS. 1 through 7. In some examples, a user device may execute a set of instructions to control the functional elements of the user device to perform the described functions. Additionally, or alternatively, the user device may perform aspects of the described functions using special-purpose hardware.

At 805, the method may include identifying one or more health-related conditions associated with the user based at least in part on PPG data collected via the wearable device, a user input received via the user device, or both. The operations of 805 may be performed in accordance with examples as disclosed herein. In some examples, aspects of the operations of 805 may be performed by a condition component 625 as described with reference to FIG. 6.

At 810, the method may include transmitting an instruction to cause a GUI of the user device to display a set of instructions for performing a wellness routine configured to alleviate the one or more health-related conditions, wherein the wellness routine is determined based at least in part on the one or more health-related conditions and the PPG data associated with the user, and wherein the set of instructions comprise one or more instructions for using one or more external devices in accordance with the wellness routine. The operations of 810 may be performed in accordance with examples as disclosed herein. In some examples, aspects of the operations of 810 may be performed by a GUI component 630 as described with reference to FIG. 6.

At 815, the method may include receiving additional PPG data acquired by the wearable device during a first time interval that the wellness routine is performed, during a second time interval subsequent to performance of the wellness routine, or both. The operations of 815 may be performed in accordance with examples as disclosed herein. In some examples, aspects of the operations of 815 may be performed by a physiological data component 635 as described with reference to FIG. 6.

At 820, the method may include transmitting an additional instruction to cause the GUI of the user device to display information associated with the wellness routine, wherein the information comprises one or more changes between the PPG data and the additional PPG data, information associated with an additional user input received in response to the wellness routine, or both. The operations of 820 may be performed in accordance with examples as disclosed herein. In some examples, aspects of the operations of 820 may be performed by a GUI component 630 as described with reference to FIG. 6.

It should be noted that the methods described above describe possible implementations, and that the operations and the steps may be rearranged or otherwise modified and that other implementations are possible. Furthermore, aspects from two or more of the methods may be combined.

A method is described. The method may include identifying one or more health-related conditions associated with a user based at least in part on PPG data collected via the wearable device, a user input received via the user device, or both, transmitting an instruction to cause a GUI of the user device to display a set of instructions for performing a wellness routine configured to alleviate the one or more health-related conditions, wherein the wellness routine is determined based at least in part on the one or more health-related conditions and the PPG data associated with the user, and wherein the set of instructions comprise one or more instructions for using one or more external devices in accordance with the wellness routine, receiving additional PPG data acquired by the wearable device during a first time interval that the wellness routine is performed, during a second time interval subsequent to performance of the wellness routine, or both, and transmitting an additional instruction to cause the GUI of the user device to display information associated with the wellness routine, wherein the information comprises one or more changes between the PPG data and the additional PPG data, information associated with an additional user input received in response to the wellness routine, or both.

An apparatus is described. The apparatus may include a processor, memory coupled with the processor, and instructions stored in the memory. The instructions may be executable by the processor to cause the apparatus to identify one or more health-related conditions associated with a user based at least in part on PPG data collected via the wearable device, a user input received via the user device, or both, transmit an instruction to cause a GUI of the user device to display a set of instructions for performing a wellness routine configured to alleviate the one or more health-related conditions, wherein the wellness routine is determined based at least in part on the one or more health-related conditions and the PPG data associated with the user, and wherein the set of instructions comprise one or more instructions for using one or more external devices in accordance with the wellness routine, receive additional PPG data acquired by the wearable device additional physiological data during a first time interval that the wellness routine is performed, during a second time interval subsequent to performance of the wellness routine, or both, and transmit an additional instruction to cause the GUI of the user device to display information associated with the wellness routine, wherein the information comprises one or more changes between the PPG data and the additional PPG data, information associated with an additional user input received in response to the wellness routine, or both.

Another apparatus is described. The apparatus may include means for identifying one or more health-related conditions associated with a user based at least in part on PPG data collected via the wearable device, a user input received via the user device, or both, means for transmitting an instruction to cause a GUI of the user device to display a set of instructions for performing a wellness routine configured to alleviate the one or more health-related conditions, wherein the wellness routine is determined based at least in part on the one or more health-related conditions and the PPG data associated with the user, and wherein the set of instructions comprise one or more instructions for using one or more external devices in accordance with the wellness routine, means for receiving additional PPG data acquired by the wearable device during a first time interval that the wellness routine is performed, during a second time interval subsequent to performance of the wellness routine, or both, and means for transmitting an additional instruction to cause the GUI of the user device to display information associated with the wellness routine, wherein the information comprises one or more changes between the PPG data and the additional PPG data, information associated with an additional user input received in response to the wellness routine, or both.

A non-transitory computer-readable medium storing code is described. The code may include instructions executable by a processor to identify one or more health-related conditions associated with a user based at least in part on PPG data collected via the wearable device, a user input received via the user device, or both, transmit an instruction to cause a GUI of the user device to display a set of instructions for performing a wellness routine configured to alleviate the one or more health-related conditions, wherein the wellness routine is determined based at least in part on the one or more health-related conditions and the PPG data associated with the user, and wherein the set of instructions comprise one or more instructions for using one or more external devices in accordance with the wellness routine, receive additional PPG data acquired by the wearable device during a first time interval that the wellness routine is performed, during a second time interval subsequent to performance of the wellness routine, or both, and transmit an additional instruction to cause the GUI of the user device to display information associated with the wellness routine, wherein the information comprises one or more changes between the PPG data and the additional PPG data, information associated with an additional user input received in response to the wellness routine, or both.

Some examples of the method, apparatuses, and non-transitory computer-readable medium described herein may further include operations, features, means, or instructions for transmitting an instruction to cause the GUI of the user device to display a prompt including one or more candidate wellness routines configured to alleviate the one or more health-related conditions, the one or more candidate wellness routines including the wellness routine and receiving, via the user device based at least in part on displaying the prompt, a selection of the wellness routine from the one or more candidate wellness routines, wherein transmitting the instruction to cause the GUI of the user device to display the set of instructions for the wellness routine may be based at least in part on receiving the selection.

Some examples of the method, apparatuses, and non-transitory computer-readable medium described herein may further include operations, features, means, or instructions for determining one or more estimated efficacy metrics associated with the one or more candidate wellness routines, wherein the one or more candidate wellness routines may be displayed based at least in part on the one or more estimated efficacy metrics.

Some examples of the method, apparatuses, and non-transitory computer-readable medium described herein may further include operations, features, means, or instructions for identifying the one or more candidate wellness routines, identifying the one or more estimated efficacy metrics, or both, based at least in part on a plurality of additional health-related conditions associated with a plurality of additional users, and a plurality of additional wellness routines performed by the plurality of additional users.

In some examples of the method, apparatuses, and non-transitory computer-readable medium described herein, the one or more instructions for using the one or more external devices comprise an indication of one or more operational settings associated with the one or more external devices.

Some examples of the method, apparatuses, and non-transitory computer-readable medium described herein may further include operations, features, means, or instructions for determining one or more operational settings associated with the one or more external devices based at least in part on the wellness routine and transmitting a signal to the one or more external devices, an additional user device associated with the one or more external devices, or both, wherein the signal comprises an additional instruction for the one or more external devices to operate in accordance with the one or more operational settings.

In some examples of the method, apparatuses, and non-transitory computer-readable medium described herein, the one or more operational settings comprise a massage or percussive intensity, a massage or percussive pattern, a temperature, a periodicity, a voltage or current, or any combination thereof.

In some examples of the method, apparatuses, and non-transitory computer-readable medium described herein, selectively modifying one or more parameters associated with the wellness routine based at least in part on the additional PPG data, the additional user input received in response to the wellness routine, or both and transmitting an instruction to cause the GUI of the user device to display an additional set of instructions for performing the modified wellness routine.

In some examples of the method, apparatuses, and non-transitory computer-readable medium described herein, the set of instructions comprise a frequency for performing the wellness routine, a duration for performing the wellness routine, instructions for an exercise routine, instructions for a diet, instructions for performing a meditation, instructions for a supplement routine, instructions for a stretching routine, instructions for a medication routine, instructions for performing cognitive behavioral therapy, or any combination thereof.

In some examples of the method, apparatuses, and non-transitory computer-readable medium described herein, the one or more external devices comprise a massage device, a percussive therapy device, a thermally-controlled device, a weighted blanket, blue light-blocking glasses, an electroshock therapy device, a sleep mask, a cryotherapy device, a red light therapy device, an infrared sauna, a compression device, an intravenous device, an intramuscular shot, a hyperbaric oxygen therapy device, a facial procedure, or any combination thereof.

In some examples of the method, apparatuses, and non-transitory computer-readable medium described herein, the one or more health-related conditions comprise muscle pain, body tension or tightness, circulation issues, issues associated with sleep, headaches, back aches, hypertension, one or more mental health-related conditions, one or more cardiovascular conditions, or any combination thereof.

In some examples of the method, apparatuses, and non-transitory computer-readable medium described herein, the wearable device comprises a wearable ring device.

In some examples of the method, apparatuses, and non-transitory computer-readable medium described herein, the PPG data, the additional PPG data, or both, may be acquired by the wearable device based on arterial blood flow, capillary blood flow, arteriole blood flow, or a combination thereof.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A system, comprising:
a wearable device configured to acquire photoplethysmogram (PPG) data from a user using one or more light-emitting components and one or more light-receiving components;
a user device communicatively coupled with the wearable device;
one or more external devices configured to perform one or more user-perceptible actions associated with one or more wellness routines that are performable by the user; and
one or more processors communicatively coupled with at least the wearable device and the user device, the one or more processors configured to:
identify one or more health-related conditions associated with the user based at least in part on the PPG data collected via the wearable device, a user input received via the user device, or both;
transmit an instruction to cause a graphical user interface of the user device to display a set of instructions for performing a wellness routine configured to alleviate the one or more health-related conditions, wherein the wellness routine is determined based at least in part on the one or more health-related conditions and the PPG data associated with the user, and wherein the set of instructions comprise one or more instructions for using the one or more external devices in accordance with the wellness routine;
receive additional PPG data acquired by the wearable device during a first time interval that the wellness routine is performed, during a second time interval subsequent to performance of the wellness routine, or both; and
transmit an additional instruction to cause the graphical user interface of the user device to display information associated with the wellness routine, wherein the information comprises one or more changes between the PPG data and the additional PPG data, information associated with an additional user input received in response to the wellness routine, or both.

2. The system of claim 1, wherein the one or more processors are further configured to:
transmit an instruction to cause the graphical user interface of the user device to display a prompt including one or more candidate wellness routines configured to alleviate the one or more health-related conditions, the one or more candidate wellness routines including the wellness routine; and
receive, via the user device based at least in part on displaying the prompt, a selection of the wellness routine from the one or more candidate wellness routines, wherein transmitting the instruction to cause the graphical user interface of the user device to display the set of instructions for the wellness routine is based at least in part on receiving the selection.

3. The system of claim 2, wherein the one or more processors are further configured to:
determine one or more estimated efficacy metrics associated with the one or more candidate wellness routines, wherein the one or more candidate wellness routines are displayed based at least in part on the one or more estimated efficacy metrics.

4. The system of claim 3, wherein the one or more processors are further configured to:
identify the one or more candidate wellness routines, identifying the one or more estimated efficacy metrics, or both, based at least in part on a plurality of additional health-related conditions associated with a plurality of additional users, and a plurality of additional wellness routines performed by the plurality of additional users.

5. The system of claim 1, wherein the one or more instructions for using the one or more external devices comprise an indication of one or more operational settings associated with the one or more external devices.

6. The system of claim 1, wherein the one or more processors are further configured to:
determine one or more operational settings associated with the one or more external devices based at least in part on the wellness routine; and
transmit a signal to the one or more external devices, an additional user device associated with the one or more external devices, or both, wherein the signal comprises an additional instruction for the one or more external devices to operate in accordance with the one or more operational settings.

7. The system of claim 6, wherein the one or more operational settings comprise a massage or percussive intensity, a massage or percussive pattern, a temperature, a periodicity, a voltage or current, or any combination thereof.

8. The system of claim 1, wherein the one or more processors are further configured to:
selectively modify one or more parameters associated with the wellness routine based at least in part on the additional PPG data, the additional user input received in response to the wellness routine, or both; and
transmit an instruction to cause the graphical user interface of the user device to display an additional set of instructions for performing the modified wellness routine.

9. The system of claim 1, wherein the set of instructions comprise a frequency for performing the wellness routine, a duration for performing the wellness routine, instructions for an exercise routine, instructions for a diet, instructions for performing a meditation, instructions for a supplement routine, instructions for a stretching routine, instructions for a medication routine, instructions for performing cognitive behavioral therapy, or any combination thereof.

10. The system of claim 1, wherein the one or more external devices comprise a massage device, a percussive therapy device, a thermally-controlled device, a weighted blanket, blue light-blocking glasses, an electroshock therapy device, a sleep mask, a cryotherapy device, a red light therapy device, an infrared sauna, a compression device, an intravenous device, an intramuscular shot, a hyperbaric oxygen therapy device, a facial procedure, or any combination thereof.

11. The system of claim 1, wherein the one or more health-related conditions comprise muscle pain, body tension or tightness, circulation issues, issues associated with sleep, headaches, back aches, hypertension, one or more mental health-related conditions, one or more cardiovascular conditions, or any combination thereof.

12. The system of claim 1, wherein the wearable device comprises a wearable ring device.

13. The system of claim 1, wherein the PPG data, the additional PPG data, or both, is acquired by the wearable device based on arterial blood flow, capillary blood flow, arteriole blood flow, or a combination thereof.

14. A method, comprising:
identifying one or more health-related conditions associated with a user based at least in part on photoplethysmogram (PPG) data collected via a wearable device, a user input received via a user device, or both;
transmitting an instruction to cause a graphical user interface of the user device to display a set of instructions for performing a wellness routine configured to alleviate the one or more health-related conditions, wherein the wellness routine is determined based at least in part on the one or more health-related conditions and the PPG data associated with the user, and wherein the set of instructions comprise one or more instructions for using one or more external devices in accordance with the wellness routine;
receiving additional PPG data acquired by the wearable device during a first time interval that the wellness routine is performed, during a second time interval subsequent to performance of the wellness routine, or both; and
transmitting an additional instruction to cause the graphical user interface of the user device to display information associated with the wellness routine, wherein the information comprises one or more changes between the PPG data and the additional PPG data, information associated with an additional user input received in response to the wellness routine, or both.

15. The method of claim 14, further comprising:
transmitting an instruction to cause the graphical user interface of the user device to display a prompt including one or more candidate wellness routines configured to alleviate the one or more health-related conditions, the one or more candidate wellness routines including the wellness routine; and
receiving, via the user device based at least in part on displaying the prompt, a selection of the wellness routine from the one or more candidate wellness routines, wherein transmitting the instruction to cause the graphical user interface of the user device to display the set of instructions for the wellness routine is based at least in part on receiving the selection.

16. The method of claim 15, further comprising:
determining one or more estimated efficacy metrics associated with the one or more candidate wellness routines, wherein the one or more candidate wellness routines are displayed based at least in part on the one or more estimated efficacy metrics.

17. The method of claim 16, further comprising:
identifying the one or more candidate wellness routines, identifying the one or more estimated efficacy metrics, or both, based at least in part on a plurality of additional health-related conditions associated with a plurality of additional users, and a plurality of additional wellness routines performed by the plurality of additional users.

18. The method of claim 14, further comprising:
determining one or more operational settings associated with the one or more external devices based at least in part on the wellness routine; and
transmitting a signal to the one or more external devices, an additional user device associated with the one or more external devices, or both, wherein the signal comprises an additional instruction for the one or more external devices to operate in accordance with the one or more operational settings.

19. The method of claim 14, further comprising:
selectively modifying one or more parameters associated with the wellness routine based at least in part on the additional PPG data, the additional user input received in response to the wellness routine, or both; and
transmitting an instruction to cause the graphical user interface of the user device to display an additional set of instructions for performing the modified wellness routine.

20. A non-transitory computer-readable medium storing code, the code comprising instructions executable by a processor to:
identify one or more health-related conditions associated with a user based at least in part on photoplethysmogram (PPG) data collected via a wearable device, a user input received via a user device, or both;
transmit an instruction to cause a graphical user interface of the user device to display a set of instructions for performing a wellness routine configured to alleviate the one or more health-related conditions, wherein the wellness routine is determined based at least in part on the one or more health-related conditions and the PPG data associated with the user, and wherein the set of instructions comprise one or more instructions for using one or more external devices in accordance with the wellness routine;
receiving additional PPG data acquired by the wearable device during a first time interval that the wellness routine is performed, during a second time interval subsequent to performance of the wellness routine, or both; and
transmit an additional instruction to cause the graphical user interface of the user device to display information associated with the wellness routine, wherein the information comprises one or more changes between the PPG data and the additional PPG data, information associated with an additional user input received in response to the wellness routine, or both.
